(19) United States
(12) Patent Application Publication
(10) Pub. No.: US 2022/0073852 A1
(43) Pub. Date: Mar. 10, 2022

(54) POROUS MICROWELL, AND MEMBRANE COMPRISING SAME AND MANUFACTURING METHOD THEREFOR

(71) Applicant: Postech Academy-Industry Foundation, Gyeongsangbuk-do (KR)

(72) Inventors: Seong Jin LEE, Daegu (KR); Jae Seung YOON, Gyeongsangbuk-do (KR); Seong Su EOM, Busan (KR); Sang Min PARK, Gyeongsangbuk-do (KR); Dong Sung KIM, Gyeongsangbuk-do (KR)

(73) Assignee: Postech Academy-Industry Foundation, Gyeongsangbuk-do (KR)

(21) Appl. No.: 17/418,758

(22) PCT Filed: Jan. 7, 2019
(86) PCT No.: PCT/KR2019/000182

§ 371 Date: Jun. 25, 2021

(30) Foreign Application Priority Data
Dec. 28, 2018 (KR) 10-2018-0171798

(51) Int. Cl. C12M 1/32; C12M 1/12; B29C 51/08; B29C 51/42

(52) U.S. Cl. CPC C12M 23/12; B29C 51/428; B29C 51/08; C12M 25/04

(57) ABSTRACT

The porous microwell according to an exemplary embodiment of the present invention is characterized in that as a porous microwell which is a cell culture surface for culturing cells, at least one thereof is indented in a downward direction and is located within a region formed by a penetrating part of a cell culture vessel, and the porous microwell includes a plurality of pores together with a connection part formed therearound, and a first porosity formed by first pores thereof and a second porosity formed by second pores formed in the connection part are different from each other.

### TECHNICAL FIELD

The present invention relates to a porous microwell, a membrane with the porous microwell, and a method for manufacturing the same, and more specifically, the present invention relates to a porous microwell providing an environment in which a three-dimensional cell spheroid can be formed, a membrane having the same, and a method for manufacturing the same.

### BACKGROUND ART

Actual cells in the body have a three-dimensional shape and interact with the microenvironment of cells in three dimensions. When cells are cultured outside the body as a two-dimensional monolayer instead of three dimensions, it is largely lacking in similarity to cells in the body.

In order to overcome the limitation of such two-dimensional monolayer cell culture, there exists a microwell platform capable of 3D cell aggregation culture. That is, the inicrowell induces cell aggregation in a microspace partitioned by single cells, thereby inducing the formation of such 3D cell aggregation.

However, conventional microwells do not have a porous structure or are formed using plastic or the like. Accordingly, the conventional microwells have a limitation in forming 3D cell aggregation.

### DISCLOSURE

### Technical Problem

In order to solve the problems of the prior art as described above, the present invention is directed to providing a porous microwell providing an environment in which a three-dimensional cell spheroid can be formed, a membrane having the same, and a method for manufacturing the same.

However, the technical problems of the present invention are not limited to the problem mentioned above, and other problems that are not mentioned will be clearly understood by those skilled in the art from the following description.

### Technical Solution

The porous microwell according to an exemplary embodiment of the present invention for solving the above problems is a porous microwell, which is a cell culture surface for culturing cells, wherein at least one thereof is indented in a downward direction and is located within a region formed by a penetrating part of a cell culture vessel, and wherein the porous microwell includes a plurality of pores together with a connection part formed therearound, and a first porosity formed by first pores thereof and a second porosity formed by second pores formed in the connection part are different from each other.

The porous microwell may have a thinner thickness than the connection part.

A plurality of the porous microwells may be located in the region formed by the penetrating part.

In the porous microwell, a flow concentration phenomenon may occur around the porous microwell, when a fluid filled in an accommodating space accommodating the porous microwell passes from the top to the bottom.

The first porosity may be greater than the second porosity.

The first pores and the second pores may be formed in a region between a plurality of polymer fibers intertwined with each other.

The membrane according to an exemplary embodiment of the present invention is a membrane employed in a penetrating part of a cell culture vessel, including (1) one or more microwells, which are cell culture surfaces for culturing cells, to be indented in a downward direction and to be located within a region formed by the penetrating part; and (2) a connection part for connecting between the microwells.

The microwell and the connection part may include a plurality of pores, and a first porosity formed by first pores formed in the microwell and a second porosity formed by second pores formed in the connection part are different from each other.

The microwell may have a thinner thickness than the connection part.

A plurality of the microwells may be located in the region formed by the penetrating part.

In the membrane, a flow concentration phenomenon may occur around the microwell, when a fluid filled in an accommodating space accommodating the microwell passes from the top of the microwell and the connection part to the bottom.

The first porosity may be greater than the second porosity.

The cell culture vessel may include a body in which the penetrating part is formed.

The cell culture vessel may include a body and a fastening part fastened to a lower portion of the body and formed with the penetrating part.

The method for manufacturing a microwell according to an exemplary embodiment of the present invention includes (a) preparing a membrane made of a plurality of polymer fibers and including a plurality of pores formed in a region between the plurality of polymer fibers; and (b) forming one or more microwells which are indented in a downward direction and a connection part connecting between the microwells in the membrane, by performing an embossing process on the membrane using a mold in which a pattern of microwells, which are cell culture surfaces for culturing cells.

Step (b) may include forming the microwell such that the microwell can be located in a region formed by a penetrating part of a cell culture vessel.

A first porosity formed by first pores formed in the microwell and a second porosity formed by second pores formed in the connection part may be different from each other.

The mold may include a first mold having a lower portion protruding according to the pattern of the microwell, and a second mold having an upper portion which is indented according to the pattern of the microwell.

Step (b) may include performing a hot embossing process using the heated mold.

Step (b) may include heating the first mold or the second mold, or heating both the first mold and the second mold.

### Advantageous Effects

The porous microwell according to an exemplary embodiment of the present invention configured as described above, a membrane having the same, and a method for manufacturing the same provide an environment in which a three-dimensional cell spheroid can be formed, and thus, these have an advantage in that cells seated in a certain region can proliferate and differentiate more smoothly in three dimensions.

### Description of Drawings

FIG. 1 shows a cell culture vessel according to an exemplary embodiment of the present invention.

FIG. 2 is a cross-sectional diagram of one side of the cell culture vessel according to an exemplary embodiment of the present invention.

FIG. 3 shows a plate 30 according to an exemplary embodiment of the present invention.

FIG. 4 shows an enlarged diagram of the periphery of one opening 31 in FIG. 2.

FIG. 5 shows a state in which cells proliferate in a cell culture vessel according to another exemplary embodiment of the present invention.

FIG. 6 shows a body 10 and a fastening part 40 of a cell culture vessel according to another exemplary embodiment of the present invention.

FIG. 7 shows a sequence of the method for manufacturing a cell culture vessel according to another exemplary embodiment of the present invention.

FIG. 8 shows an example of S10 or S100 of the method for manufacturing a cell culture vessel according to another exemplary embodiment of the present invention.

FIG. 9 shows an example of S20 or S200 of the method for manufacturing a cell culture vessel according to another exemplary embodiment of the present invention.

FIG. 10 shows images of a membrane 20 formed by the method for manufacturing a cell culture vessel according to an exemplary embodiment of the present invention.

### EXPLANATION OF REFERENCE NUMERALS

| | |
|---|---|
| 10: Body | 11: Inlet |
| 20: Membrane | 21: Well |
| 22: Connection part | 23: Fixing part |
| 30: Plate | 31: Opening |
| 40: Fastening part | 50: Electrolyte solution |
| 60: Electrolyte container | 70: Electrospinning machine |
| 71: Metal needle | |

### Modes of the Invention

The above objects and means of the present invention and effects thereof will become more apparent through the following detailed description in relation to the accompanying drawings, and accordingly, those of ordinary skill in art to which the present invention pertains may easily practice the technical spirit of the present invention. In addition, in describing the present invention, when it is determined that a detailed description of a known technology related to the present invention may unnecessarily obscure the gist of the present invention, detailed description thereof will be omitted.

The terms used in the present specification are for describing exemplary embodiments and are not intended to limit the present invention. In the present specification, the singular form also includes the plural form in some cases unless specifically stated in the phrase. In the present specification, terms such as “comprise”, “provide with”, “include”, or “have” do not exclude the presence or addition of one or more other constituent elements other than the mentioned constituent elements.

In the present specification, terms such as “or”, “at least one”, and the like may represent one of words listed together, or a combination of two or more. For example, “or B” and “at least one of B” may include only one of A or B, and may include both A and B.

In the present specification, the description following “for example” may not exactly match the information presented, such as the recited characteristics, variables, or values, and the exemplary embodiments of the invention according to various examples of the present invention should not be limited to effects such as variations including tolerances, measurement errors, limitations of measurement accuracy, and other commonly known factors.

In the present specification, when a component is described as being ‘connected’ or ‘joined’ to another component, it may be directly connected or joined to the other component, but it should be understood that other components may be present in the middle. On the other hand, when a component is referred to as being ‘directly connected’ or ‘directly joined’ to another component, it should be understood that there is no other component in the middle.

In the present specification, when a component is described as being ‘on’ or ‘adjacent’ of another component, it may be directly in contact with or connected to another component, but it should be understood that another component may be present in the middle. On the other hand, when a component is described as being ‘directly above’ or ‘directly adjacent’ of another component, it may be understood that another component does not exist in the middle. Other expressions describing the relationship between components, for example, ‘between’ and ‘directly between’ can be interpreted in this way.

In the present specification, terms such as ‘first’ and ‘second’ may be used to describe various components, but the corresponding components should not be limited by the above terms. In addition, the above terms should not be interpreted as limiting the order of each component, and may be used for the purpose of distinguishing one component from another component. For example, the ‘first component’ may be named as the ‘second component’, and similarly, the ‘second component’ may also be named as the ‘first component.’

Unless otherwise defined, all terms used in the present specification may be used with meanings that can be commonly understood by those of ordinary skill in the art to which the present invention pertains. In addition, terms defined in a commonly used dictionary are not interpreted ideally or excessively unless explicitly defined specifically.

Hereinafter, a preferred exemplary embodiment according to the present invention will be described in detail with reference to the accompanying drawings.

The porous microwell according to an exemplary embodiment of the present invention and the membrane having the same may be employed in a cell culture vessel, but are not limited thereto. However, for convenience of description, an exemplary embodiment in which these configurations are employed in a cell culture vessel will be described below.

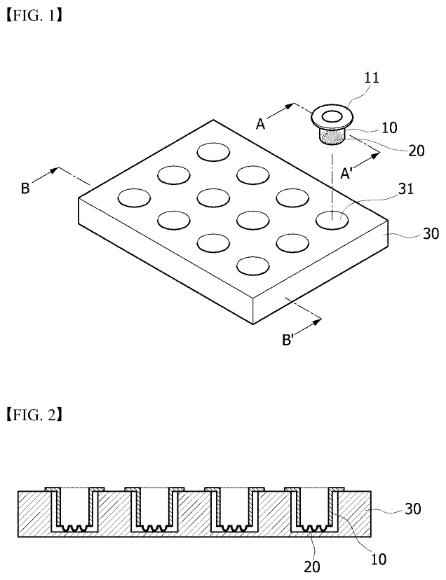

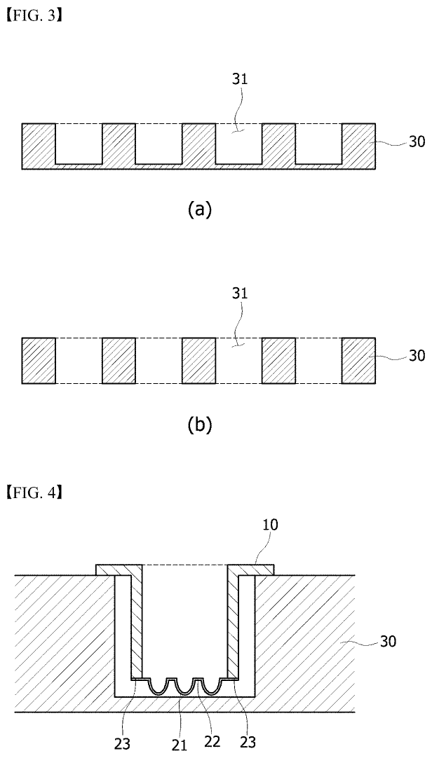

FIG. 1 shows a cell culture vessel according to an exemplary embodiment of the present invention, and FIG. 2 is a cross-sectional diagram of one side of the cell culture vessel according to an exemplary embodiment of the present invention. In this case, FIG. 2 shows a cross section of one side cutting A and A′ in FIG. 1. FIG. 3 shows a plate 30 according to an exemplary embodiment of the present invention.

The cell culture vessel according to an exemplary embodiment of the present invention may include a body 10 and a membrane 20, as illustrated in FIGS. 1 and 2, and may further include a plate 30. However, hereinafter, for convenience of description, the plate 30 will be described first, and then, the body 10 and the membrane 20 will be described in order.

The plate 30 has one or more openings 31 having an opening shape, and the body 10 may be inserted and mounted into the opening 31. In this case, the opening 31 may have an open shape with a lower portion closed and an upper portion open, as illustrated in FIG. 3(a), or a penetrating shape penetrating the plate 30, as illustrated in FIG. 3(b). When the opening 31 has a penetrating shape, the cell culture vessel according to an exemplary embodiment of the present invention may further include a cover container having an accommodating space with an open upper portion and mounting the plate 30 in the accommodating space.

In particular, when a plurality of openings 31 are provided in the plate 30, since each opening 31 is arranged to be separated from each other, the cell culture vessel according to an exemplary embodiment of the present invention may block the influence between samples of each opening 31 during cell culture, and accordingly, multiple independent experimental data may be derived using one plate 30.

As illustrated in FIGS. 1 and 2, the body 10 includes a spacer formed at one end to have a side wall, an inlet 11 formed at the other end, and a penetrating part penetrating the spacer and the inlet 11, respectively.

The spacer of the body 10 may be inserted into the opening 31 of the plate 30. In this case, the body 10 may include one insertion part or may include a plurality of insertion parts. That is, when including one insertion part, the body 10 may be inserted into one opening 31 of the corresponding insertion part. In addition, when a plurality of insertion parts are included, each insertion part may be inserted corresponding to a plurality of openings 31 in the body 10. In this case, when a plurality of insertion parts are included, the body 10 has a structure in which each insertion part is connected to each other, and each insertion part may be inserted corresponding to each opening 31.

While FIGS. 1 to 6 illustrate a body 10 including one insertion part, the present invention is not limited thereto, and the contents of the present invention may certainly be applied even when the body 10 includes a plurality of spacers and penetrating parts.

When the spacer of the body 10 is inserted into the opening 31, the spacer of the body 10 is located at the bottom, and the inlet 11 of the body 10 is located at the top. The spacer of the body 10 may have a vertical shape having a constant width from one end to the other end, a funnel shape in which the width gradually expands from one end to the other end, or a form in which a vertical form and a funnel form are combined. The penetrating part of the body 10 may be formed in various shapes such as a circular shape, a polygonal cross section, or the like, and various sizes thereof may be formed.

FIG. 4 shows an enlarged diagram of the periphery of one opening 31 in FIG. 2.

In particular, when the spacer of the body 10 is mounted on the opening 31 of the plate 30, as illustrated in FIG. 4, it is preferable that the inlet 11 of the body 10 has a cross section that is wider than the inlet of the spacer and the opening 31 of the body, in order that one end of the spacer of the body 10 is located at a certain distance from the bottom surface of a cell culture vessel. Accordingly, in the space between the spacer of the body 10 and the bottom surface of the cell culture vessel, a passage for a fluid having nutrients for supplying the cells may be formed. In this case, the bottom surface of the cell culture vessel may be the bottom surface of the opening in the case of an open plate, and may be the bottom surface of the cover container in the case of a penetrating plate.

The membrane 20 is a layer that provides a cell culture surface on which cells are cultured, and is employed in the penetrating part on the side of the spacer of the body 10. For example, the membrane 20 may be formed through electrospinning to cover one end of the spacer of the body 10. In this case, the membrane 20 may be formed by randomly intertwining a plurality of polymer nanofibers, or may be formed by molding a polymer synthetic resin. For example, each polymer nanofiber may have a diameter of 1 nm or more to less than 1,000 nm. As it is made of a plurality of polymer nanofibers, the membrane 20 has a structure similar to that of the basement membrane in a living body, thereby providing a blood flow environment in the living body.

For example, the polymer nanofiber or polymer synthetic resin may include at least one or more of a thermoplastic resin, a thermosetting resin, an elastomer, and a biopolymer. For example, polymer nanofibers or synthetic resins may include at least one or more of polycaprolactone, polyurethane, polyvinylidene fluoride (PVDF), polystyrene, collagen, gelatin, and chitosan.

The membrane 20 may include a porous microwell 21, a connection part 22, and a fixing part 23. In this case, the microwell 21, the connection part 22, and the fixing part 23 have a structure in which these are connected to each other by intertwining a plurality of polymer nanofibers.

The microwell 21 is a region that acts as a cell culture surface, and is formed to be indented in a downward direction. That is, by this indented shape, cells are easily seated in the microwell 21 and may be stably proliferated in the microwell 21 regardless of the movement of a fluid. In this case, at least one of the microwells 21 is located in a region formed by the penetrating part of the body 10. That is, when viewed from the top or bottom, the microwell 21 is smaller in size than the penetrating part of the body 10 and is included in the region formed by the penetrating part.

That is, as the microwell 21, which is a cell culture surface, is formed in an indented shape, the membrane 20 may attach the cells to the cell culture surface more intensively and stably and increase the area of the cell culture surface such that it may improve the adhesion efficiency of cells. In addition, unlike conventional cell culture vessels having a cell culture surface of conventionally flat shapes, the body 10 of the cell culture vessel according to an exemplary embodiment of the present invention may form a three-dimensional cell spheroid by providing a cell culture surface in a three-dimensional shape to culture cells in a three-dimensional structure environment as in the living body. However, when a plurality of microwells 21 are located within a region formed by the penetrating part of the body 10, the spacer of the body 10 has a plurality of cell culture surfaces such that cell adhesion efficiency may be further improved.

The connection part 22 is a region formed around the microwells 21 to connect between the microwells 21 and may have a flat shape. In particular, the connection part 22 may be thicker than the microwell 21. This corresponds to a region in which the microwell 21 extends in a lower indented shape among the membranes 20 by an embossing process to be described below such that it becomes thinner than the original, whereas the connection part 22 corresponds to a region that is not extended such that the original thickness is maintained.

The fixing part 23 is an area fixed to the edge of one end of the spacer of the body 10. In particular, the fixing part 23 may have a thinner thickness and a lower density than the connection part 22. This is because the membrane 20 may be formed through electrospinning using an electrolyte solution. That is, since the microwell 21 and the connection part 22 correspond to a region generated at a location where the electrolyte solution is contained during electrospinning, a greater number of polymer nanofibers are formed than the fixing part 23, and thus, it may be formed with a higher density and thicker thickness than the fixing part 23.

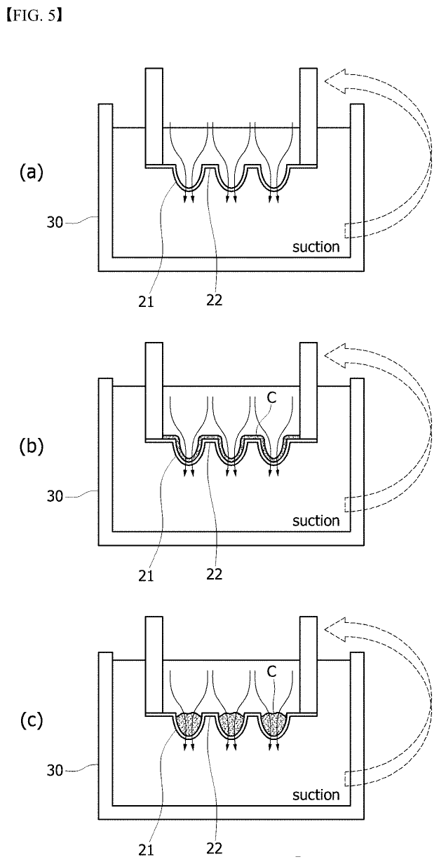

FIG. 5 shows a state in which cells proliferate in a cell culture vessel according to another exemplary embodiment of the present invention. That is, FIGS. 5(a), 5(b), and 5(c) sequentially show a state in which cells proliferate over time under a fluid concentration phenomenon.

Meanwhile, the membrane 20 includes a plurality of pores formed in a region between the polymernanofibers. In this case, the pores may have a size of several um to tens of μm. That is, the membrane 20 may act as a selective permeable membrane that does not allow single cells to permeate but selectively permeates other materials due to the pores, and thus may serve as a material transfer barrier and passage.

The first porosity formed by the first pores formed in the microwell 21 and the second porosity formed by the second pores formed in the connection part 22 may be different from each other. In this case, the porosity may be a ratio of the pore area existing in the unit area. In particular, the first porosity may be greater than the second porosity. This is because the region of the membrane 20 corresponding to the microwell 21 is stretched into a lower indented shape by the embossing process, and a phenomenon occurs in which a number of portions blocked by the intertwined polymer nanofibers are opened or the area of the already opened portion is widened. However, these two porosities are described in order to have a difference between the first porosity and the second porosity, but the present invention is not limited to having only two types of these porosities. That is, the present invention may have various porosities in addition to the first porosity and the second porosity.

According to the difference in such porosities, as illustrated in FIG. 5, while a fluid concentration phenomenon occurs in a region around the microwell 21, cell proliferation in the microwell 21 may occur more actively. This is because as the region has a high porosity, the material permeability is higher according to Darcy's equation.

For cell culture, a fluid (e.g., a mixture of cell culture solution, distilled water, PBS solution, etc.) is filled in the opening 31 of the plate 30, and this fluid should be periodically replaced by using a spuit or the like. In this case, the fluid may be replaced by a method in which the fluid is suctioned and discharged from the outside of the body 10 and a new fluid is supplied to the inside of the body 10 at the same time. This is because when the fluid is suctioned and discharged from the inside of the body 10, there is a risk that the cells that are seated in the microwell 21 and proliferating and differentiating may be adversely affected or the corresponding cells may be discharged together.

In the above-described fluid replacement process, the fluid filled in the accommodating space accommodating the microwell 21 passes from the top of the microwell 21 and the connection part 22 to the bottom. In this case, since the microwell 21 has a greater porosity than the connection part 22, as illustrated in FIG. 5, more fluid permeates through the microwell 21 compared to the connection part 22. Accordingly, a phenomenon in which the fluid moves more intensively, that is, a fluid concentration phenomenon, occurs in the vicinity of the microwell 21 than in the vicinity of the connection part 22.

When such a fluid concentration phenomenon occurs, oxygen and nutrients contained in the fluid may be more smoothly supplied to cells proliferating and differentiating within the microwell 21, and thus, cell proliferation and differentiation may be further promoted. In addition, due to this fluid concentration phenomenon, a phenomenon in which cells are collected into the microwell 21 also occurs. That is, a plurality of regions having a difference in porosity are provided, but as the microwell 21, which is a cell culture surface among the corresponding regions, is formed to have a higher porosity than the connection part 22, the membrane 20 may increase the efficiency of proliferation and differentiation of cells in the microwell 21.

When a plurality of openings 31 are provided in the plate 30, the cell culture vessel according to the present invention may derive multiple independent experimental data tested in a three-dimensional structure environment, such as in the living body, using one plate 30.

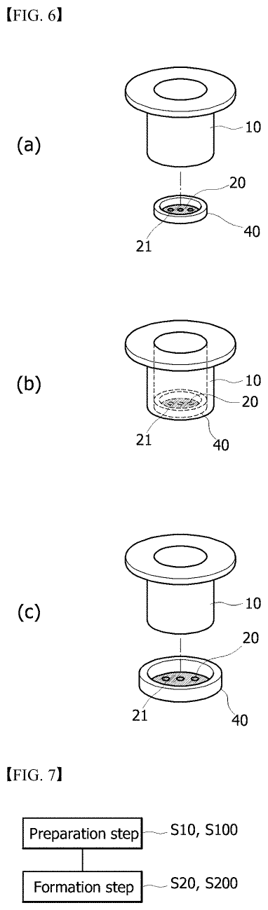

FIG. 6 shows a body 10 and a fastening part 40 of a cell culture vessel according to another exemplary embodiment of the present invention.

Meanwhile, as illustrated in FIG. 6, the cell culture vessel according to another exemplary embodiment of the present invention may further include a fastening part 40 having one end and the other end passing through and fastened to one end of the spacer of the body 10.

In this case, the fastening part 40 may include one penetrating part or may include a plurality of penetrating parts. That is, in the case of including one penetrating part, the fastening part 40 may be fastened to each spacer of the body 10 and may be formed in a ring shape. In addition, when a plurality of penetrating parts are included, the fastening part 40 may be fastened with each penetrating part corresponding to each spacer of the body 10. While FIG. 6 illustrates a case where the fastening part 40 includes one penetrating part, the present invention is not limited thereto, and the contents of the present invention may certainly be applied even when the fastening portion 40 includes a plurality of penetrating parts.

When the fastening part 40 is further included, the membrane 20 is not provided at one end of the spacer of the body 10, and the membrane 20 may be provided at one end of the fastening part 40, or the membrane 20 may be provided together at one end of the spacer of the body 10 and one end of the fastening part 40. When the fastening part 40 is fastened to the spacer of the body 10, the penetrating part of the fastening part 40 and the penetrating part of the body 10 are connected to each other.

The fastening part 40 may be provided in a form that is detachable (mounted and separated) at one end of the spacer of the body 10. For example, a screw thread may be formed inside or outside the fastening part 40, and a screw thread corresponding to the screw thread of the fastening part 40 may be formed outside or inside one end of the body. In addition, as illustrated in FIG. 6(b), the fastening part 40 may be fitted and coupled to the inner circumferential surface of the penetrating part of one end of the spacer of the body 10, or as illustrated in FIG. 6(c), it may be fitted and coupled to the outer circumferential surface of one end of the spacer the body 10.

The membrane 20 provided at one end of the fastening part 40 is the same except that the spacer of the body 10 is replaced with the fastening part 40 in the membrane 20 provided at one end of the spacer of the body 10 described above. That is, the position of the membrane 20 provided at one end of the fastening part 40 is only changed from one end of the spacer of the body 10 to one end of the fastening part 40. Accordingly, detailed descriptions of the membrane 20 provided at one end of the fastening part 40 will be omitted below, and it will be replaced with a description of the membrane 20 provided at one end of the spacer of the body 10 described above.

Hereinafter, the method for manufacturing a cell culture vessel according to an exemplary embodiment of the present invention will be described. The method for manufacturing such a cell culture vessel includes a method for manufacturing a microwell 21 or a membrane 20.

FIG. 7 shows a sequence of the method for manufacturing a cell culture vessel according to another exemplary embodiment of the present invention.

As illustrated in FIG. 7, the method for manufacturing a cell culture vessel according to an exemplary embodiment of the present invention includes preparation steps S10, S100 and formation steps S20, S200. In this case, S10 and S20 are the manufacturing methods of the body 10 and the membrane 20 described above, and S100 and S200 are the manufacturing methods of the body 10, the membrane 20, and the fastening part 40 described above.

S10 is a step of preparing the body 10 and the membrane 20. In addition, S100 is a step of preparing the body 10, the membrane 20, and the fastening part 40. In this case, since the body 10, the membrane 20, and the fastening part 40 are the same as those described above according to FIGS. 1 to 6, descriptions thereof will be omitted below. However, the membrane 20 may be formed through an electrospinning method using an electrolyte solution, and the electrospinning method using an electrolyte solution will be described below.

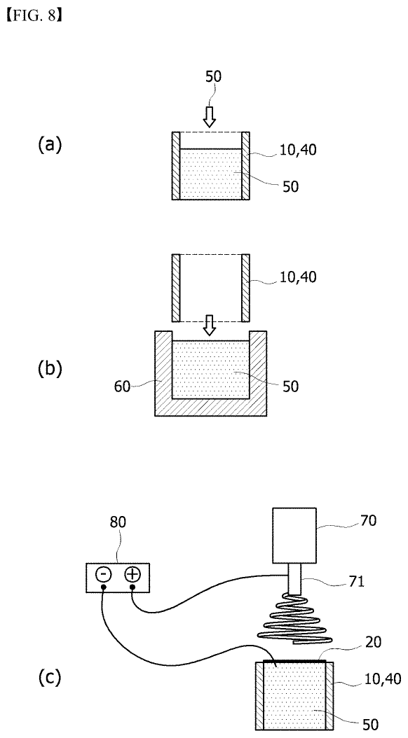

FIG. 8 shows an example of S10 or S100 of the method for manufacturing a cell culture vessel according to another exemplary embodiment of the present invention.

The electrospinning method using an electrolyte solution is to form the membrane 20 so as to cover one end of the spacer of the body 10 or the fastening part 40, and may be performed inside a chamber. In this case, the chamber is a space in which work is performed, and when the membrane 20 is formed, leakage of the polymer solution to the outside may be prevented. Hereinafter, the electrospinning method using an electrolyte solution forming the membrane 20 at one end of the spacer of the body 10 is referred to as “a first electrospinning method”, and the electrospinning method using an electrolyte solution forming the membrane 20 at one end of the fastening portion 40 is referred to as “a second electrospinning method.” First, the first electrospinning method will be described.

The first electrospinning method may sequentially include an electrolyte filling step, a voltage application step, and a membrane formation step.

That is, as illustrated in FIG. 8(a), the electrolyte filling step is a step of filling an electrolyte solution 50 in the spacer of the body 10 formed such that one end and the other end pass through. In this case, the body 10 is disposed such that one end of the spacer faces upward and the other end is blocked. Afterwards, the electrolyte solution 50 is filled into one end of the spacer of the body 10. In this case, a stopper is provided to close the inlet 11 of the body 10, and the corresponding stopper may be provided with an electrode for applying a voltage to the electrolyte solution 50. That is, the electrode is formed to pass through the stopper, and may be connected to the electrolyte solution 50 filled in the spacer of the body 10.

Alternatively, as illustrated in FIG. 8(b), in the electrolyte filling step, the spacer of the body 10 may be disposed in the electrolyte container 60 filled with the electrolyte solution 50, but the electrolyte solution 50 may be filled in the penetrating part of the body 10 by disposing one end of the spacer of the body 10 towards the top. When the spacer of the body 10 is disposed in the accommodating space of the electrolyte container 60, a pressure to press the surface of the electrolyte solution 50 is generated while the spacer of the body 10 is in contact with the surface of the electrolyte solution 50, and by this pressure, the electrolyte solution 50 is filled in the spacer of the body 10. In this case, in order that the pressure on the surface of the electrolyte solution 50 may be better generated, the accommodating space of the electrolyte container 60 may be formed to match the shape of the spacer of the body 10.

Since the electrolyte solution 50 has conductivity, when a voltage is applied in the voltage application step, it becomes (−) charge, attracting particles with (+) charge by electrical attraction, and accordingly, particles with (+) charge may be accumulated on top of the electrolyte. The electrolyte solution 50 is classified into a strong electrolyte and a weak electrolyte according to the degree of dissociation. The degree of dissociation is different depending on the solvent.

For example, as the electrolyte solution 50, a solution obtained by nixing potassium chloride and distilled water at a ratio of 3% mol may be used. In addition, materials dissolved in water or an organic solvent (ethanol and methanol) and exhibit electrical conductivity higher than 1 mS/cm may be used as the electrolyte solution 50. In addition, materials at concentrations dissolved in water and having a relative dielectric constant higher than 80 F/m may be used as the electrolyte solution 50.

Afterwards, as illustrated in FIG. 8(c), the voltage application step is a step of applying a voltage between an electrolyte solution 50 and a metal needle 71 of an electrospinning machine 70. In this case, the voltage is supplied through a power supply, and the structure of the membrane 20 formed in the membrane formation step formed in accordance with changes in the intensity of the applied voltage may be changed.

That is, an electric field is formed between the electrolyte solution 50 and the metal needle 71 of the electrospinning machine 70, and if the strength of the electric field formed at this time is too low, the polymer solution is not continuously discharged. Thus, not only is it difficult to manufacture polymer nanofibers having a uniform thickness, but also it may be difficult for the manufactured polymer nanofibers to be smoothly focused on the electrolyte solution 50. Conversely, when the strength of the electric field is too high, it may be difficult to have a normal shape because the polymer fibers are not accurately seated on the upper surface of the electrolyte solution 50. In consideration of this content, the strength of the voltage applied to the electrolyte solution 50 and the metal needle 71 of the electrospinning machine 70 may range from 5 kV to 30 kV.

A negative (−) voltage may be applied to the electrolyte solution 50, and a positive (+) voltage may be applied to the metal needle 71. Accordingly, the electrolyte solution 50 has a negative (−) charge, and the polymer solution spun in the membrane formation step has a positive (+) charge.

Afterwards, as illustrated in FIG. 8(c), the membrane formation step is a step in which a polymer solution is spun to the spacer of the body 10 through the electrospinning machine 70 while a voltage is applied to form the membrane 20. In this case, the membrane 20 is formed in a network shape in which a plurality of polymer nanofibers are randomly intertwined due to the high degree of freedom of the electrolyte solution 50.

Meanwhile, the electrospinning machine 70 is a device for supplying a polymer solution. That is, the electrospinning machine 70 may store the polymer solution to have an appropriate viscosity for electrospinning, and then discharge the polymer solution through the metal needle 71. In this case, the discharged polymer solution may be scattered and cured at the same time to form polymer nanofibers.

The metal needle 71 is a configuration to discharge a polymer solution. As it is made of a metal material, the metal needle 71 is easily connected to the power supply, and it is possible to improve the charge charging efficiency of the polymer solution discharged when a voltage is applied from the power supply. In particular, the metal needle 71 is located at an upper portion spaced apart from the spacer of the body 10, but while the discharging end thereof is disposed to face the spacer of the body 10, the polymer solution may be spun.

For example, the electrospinning machine 70 may be composed of a syringe, a syringe pump, and a metal needle 71. That is, the polymer solution may be put into a syringe, and the polymer solution may be discharged into the air by the metal needle 71 through the power of the syringe pump. In this case, the metal needle 71 may use a 23 Gauge needle, but the size of may vary depending on the polymer solution. In particular, the polymer solution may be spun at a discharge rate of 0.01 mL/h to 3 mL/h such that polymer fibers may be placed on the surface of the electrolyte solution 50 while maintaining the surface shape of the electrolyte solution 50.

When the polymer solution is spun in the above-described voltage application range (5 kV to 30 kV) and discharge rate range (0.01 mL/h to 3 mL/h), the polymer nanofibers may be formed to have a diameter of 10 nm to 900 nm.

For example, as the polymer solution, a solution having a concentration of 5% to 25% in which polycaprolactone is mixed with a solution of chloroform and methanol mixed at a mass ratio of 1:1 may be used. In addition, after mixing acetone and dimethylformamide at a volume ratio of 3:7, a solution having a concentration of 25% to 30% in which polvvinylidene fluoride (PVDF) is mixed may be used as a polymer solution. Other than the above, a polymer solution may be prepared using polystyrene, polycarbonate, a collagen/polycarbonate blending solution, gelatin, and the like.

In particular, in the membrane formation step, the electrical attraction generated between the penetrating part on the side of the spacer of the body 10 filled with the electrolyte solution 50 and the polymer solution is constant, but it is larger than the electric attraction generated between the rim of the spacer of the body 10 and the polymer solution. Accordingly, the region of the membrane 20 (hereinafter, referred to as “a penetrating part region”) accumulated in the penetrating part on the side of the spacer of the body 10 filled with the electrolyte solution 50 is constant, but it has a relatively large density and thickness.

On the other hand, the size of the electrical attraction generated between the rim of the spacer of the body 10 and the polymer solution is smaller than the size of the electrical attraction generated between the penetrating part of the spacer of the body 10 and the polymer solution, and it becomes smaller as it is further away from the penetrating part of the spacer of the body 10. Accordingly, the fixing part 23, which is the membrane 20 accumulated on the rim of the spacer of the body 10, is not constant, but has a relatively small density and thickness.

The membrane formation step may further include controlling at least one of thickness, porosity, and transparency of the membrane 20 formed at one end of the spacer of the body 10 by adjusting the spinning time of the electrospinning machine 70. That is, as the spinning time of the electrospinning machine 70 increases, the amount of polymer nanofibers to be accumulated increases. Accordingly, as the thickness of the membrane 20 formed on one end of the spacer of the body 10 increases, the porosity and transparency thereof decrease.

In addition, the membrane formation step may further include adjusting the diameter of the polymer nanofibers of the membrane 20 formed by adjusting the concentration of the polymer solution. That is, as the concentration of the polymer solution increases, the viscosity thereof increases such that the diameter of the polymer nanofibers of the membrane 20 formed at one end of the spacer of the body 10 increases.

Next, a second electrospinning method will be described.

The second electrospinning method, like the first electrospinning method, includes an electrolyte filling step, a voltage application step, and a met brane formation step, and may further include a fastening part-fastening step. In this case, the electrolyte filling step, the voltage application step, and the membrane formation step are the same as described above except that the spacer of the body 10 is replaced with the fastening part 40 in the first electrospinning method. Accordingly, detailed descriptions of the electrolyte filling step, voltage application step, and membrane formation step of the second electrospinning method are omitted below, and these will be replaced with descriptions of the electrolyte filling step, voltage application step, and membrane formation step of the first electrolyte electrospinning method described above.

That is, in the second electrospinning method, the membrane 20 may be formed at one end of the fastening part 40 through an electrolyte filling step, a voltage application step, and a membrane formation step. Afterwards, the fastening part-fastening step is a step of fastening the fastening part 40 on which the membrane 20 is formed at one end of the spacer of the body 10 formed such that one end and the other end pass through. For example, the fastening part-fastening step may be performed by a transfer device that transfers the spacer of the body 10 or the fastening part 40 to fasten the spacer of the body 10 and the fastening part 40.

However, the first electrospinning method and the second electrospinning method may further include a step of cutting the membrane 20 formed according to the shape of the spacer of the body 10 or the fastening part 40, after forming the membrane 20.

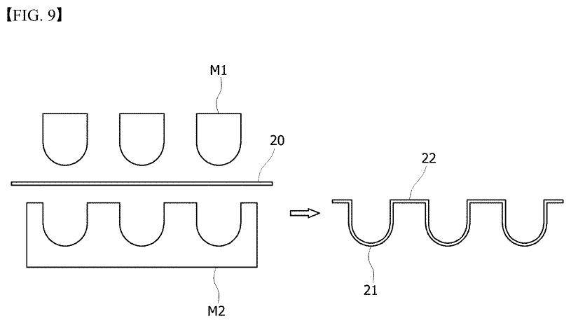

FIG. 9 shows an example of S20 or S200 of the method for manufacturing a cell culture vessel according to another exemplary embodiment of the present invention.

S20 is a step of performing an embossing process on the membrane 20 formed on the spacer of the body 10. In addition, S200 is a step of performing an embossing process on the membrane 20 formed on the fastening part 40.

That is, in S20 or S200, as illustrated in FIG. 9, an embossing process is performed on the membrane 20 prepared in S10 or S100 using a mold M in which the pattern of the microwell 21 is formed. In this case, the embossing process is a process of forming a pattern corresponding to the pattern of the mold M on the membrane 20 by pressing the membrane 20 with the mold M. That is, as a result of the embossing process, the microwell 21 and the connection part 22 may be formed in the membrane 20.

The embossing process may be a hot embossing process in which the membrane 20 is pressed with the heated mold M, but is not limited thereto. However, in the case of using a hot embossing process, the result of S20 may be derived more quickly.

The mold M may include a first mold M1 whose lower portion protrudes according to the pattern of the microwell 21 and a second mold M2 whose upper portion is indented according to the pattern of the microwell 21. That is, the membrane 20 is placed between the first mold M1 and the second mold M2, and the microwell 21 and the connection part 22 may be formed in the membrane 20 by pressing and combining the first mold M1 and the second mold M2 and then separating. When combined, the protruding portion of the first mold M1 contacts one surface of the membrane 20, and the indented portion of the second mold M2 contacts the other surface of the membrane 20.

In the case of the hot embossing process, either of the first mold M1 and the second mold M2 may be heated, or both the first mold M1 and the second mold M2 may be heated before combining. However, since the protruding shape of the first mold M1 has more influence on the formation of the microwell 21, it may be preferable to heat and use only the first mold M1. In addition, the temperature of the heated first mold M1 or second mold M2 may be preferably lower than the meltingpoint of the polymer nanofibers forming the membrane 20.

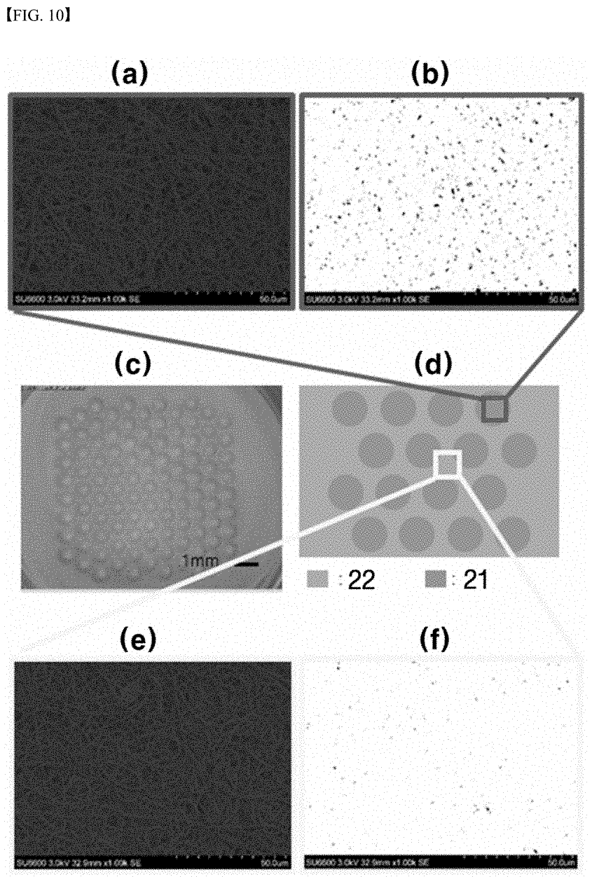

FIG. 10 shows images of a membrane 20 formed by the method for manufacturing a cell culture vessel according to an exemplary embodiment of the present invention. In this case, FIG. 10(c) shows a plan image of the membrane 20, and FIG. 10(d) shows a plan view of the microwell 21 and the connection part 22, which are illustrated by enlarging FIG. 10(c). In addition, FIG. 10(a) shows an enlarged image of the microwell 21, and FIG. 10(b) shows first pores formed in the microwell 21 of FIG. 10(a). In addition, FIG. 10(e) shows an enlarged image of the connection part 22, and FIG. 10(f) shows second pores formed in the connection part 22 of FIG. 10(e).

As illustrated in FIGS. 10(a) and 10(e), it can be confirmed that the membrane 20 formed by the method for manufacturing a cell culture vessel according to an exemplary embodiment of the present invention is formed in a network shape in which a plurality of polymer nanofibers are intertwined. In addition, referring to FIGS. 10(b) and 10(f), in the membrane 20 formed by the method for manufacturing a cell culture vessel according to an exemplary embodiment of the present invention, it can be confirmed that the number of first pores formed in the microwell 21 is greater than that of second pores formed in the connection part 22. In this case, it can be confirmed that the first porosity is increased by about 10 times or more compared to the second porosity.

Although specific exemplary embodiments have been described in the detailed description of the present invention, various modifications are possible without departing from the scope of the present invention. Therefore, the scope of the present invention is not limited to the described exemplary embodiments, and should be defined by the claims to be described below and equivalents to the claims.

### INDUSTRIAL APPLICABILITY

The present invention relates to a porous microwell, a membrane having the same, and a method for manufacturing the same, and the present invention provides a porous microwell which provides an environment in which a three-dimensional cell spheroid can be formed such that cells seated in a certain region can proliferate and differentiate more smoothly in three dimensions, a membrane having the same, and a method for manufacturing the same, and thus, it has industrial applicability.

1. A porous microwell, which is a cell culture surface for culturing cells, wherein at least one thereof is indented in a downward direction and is located within a region formed by a penetrating part of a cell culture vessel, and
wherein the porous microwell comprises a plurality of pores together with a connection part formed therearound, and a first porosity formed by first pores thereof and a second porosity formed by second pores formed in the connection part are different from each other.

2. The porous microwell of claim 1, wherein the porous microwell has a thinner thickness than the connection part.

3. The porous microwell of claim 1, wherein a plurality of the porous microwells are located in the region formed by the penetrating part.

4. The porous microwell of claim 1, wherein a flow concentration phenomenon occurs around the porous microwell, when a fluid filled in an accommodating space accommodating the porous microwell passes from the top to the bottom.

5. The porous microwell of claim 4, wherein the first porosity is greater than the second porosity.

6. The porous microwell of claim 1, wherein the first pores and the second pores are located in a region between a plurality of polymer fibers intertwined with each other.

7. A membrane employed in a penetrating part of a cell culture vessel, the membrane comprising:
one or more microwells, which are cell culture surfaces for culturing cells, to be indented in a downward direction and to be located within a region formed by the penetrating part; and
a connection part for connecting between the microwells,
wherein the microwell and the connection part comprise a plurality of pores, and a first porosity formed by first pores formed in the microwell and a second porosity formed by second pores formed in the connection part are different from each other.

8. The membrane of claim 7, wherein a flow concentration phenomenon occurs around the microwell, when a fluid filled in an accommodating space accommodating the microwell passes from the top of the microwell and the connection part to the bottom.

9. The membrane of claim 7, wherein the first porosity is greater than the second porosity.

10. The membrane of claim 7, wherein the cell culture vessel comprises a body in which the penetrating part is formed.

11. The membrane of claim 7, wherein the cell culture vessel comprises a body and a fastening part fastened to a lower portion of the body and formed with the penetrating part.

12. A method for manufacturing a porous microwell, comprising:
(a) preparing a membrane made of a plurality of polymer fibers and comprising a plurality of pores formed in a region between the plurality of polymer fibers; and
(b) forming one or more microwells which are indented in a downward direction and a connection part connecting between the microwells in the membrane, by performing an embossing process on the membrane using a mold in which a pattern of microwells which are culture surfaces for culturing cells,
wherein Step (b) comprises forming the microwell such that the microwell can be located in a region formed by a penetrating part of a cell culture vessel, and
wherein a first porosity formed by first pores formed in the microwell and a second porosity formed by second pores formed in the connection part are different from each other.

13. The method of claim 12, wherein the mold comprises:
a first mold having a lower portion protruding according to the pattern of the microwell; and
a second mold having an upper portion which is indented according to the pattern of the microwell.

14. The method of claim 12, wherein Step (b) comprises performing a hot embossing process using the heated mold.

15. The method of claim 14, wherein Step (b) comprises heating the first mold or the second mold, or heating both the first mold and the second mold.
